(19) United States
(12) Patent Application Publication
(10) Pub. No.: US 2002/0143387 A1
(43) Pub. Date: Oct. 3, 2002

(54) Stent repositioning and removal

(76) Inventors: Roy M. Soetikno, Union City, CA (US); Shai Friedland, Palo Alto, CA (US)

Correspondence Address: PAMELA J. SHERWOOD, Bozicevic, Field and Francis LLP, Suite 200, 200 Middlefield Road, Menlo Park, CA, 94025, US

(21) Appl. No.: 09/900,320

(22) Filed: Jul. 6, 2001

Related U.S. Application Data
(60) Provisional application No. 60/279,224, filed on Mar. 27, 2001.

(51) Int. Cl. A61F 2/06

(52) U.S. Cl. 623/1.15

(57) ABSTRACT

A stent removal system. Surgical devices and methods are provided for a system that allows the repositioning or removal of luminal stents. A conventional stent is modified by the addition of a tightening drawstring with a loop or region for grasping. When the stent is inserted within the body, the drawstring is slack and does not constrict the stent. When the drawstring is tightened, for example by pulling on the region for grasping, the circumference of the stent at the terminus is decreased, allowing the stent to be repositioned or removed without trauma to the organ.

### BACKGROUND OF THE INVENTION

Metallic stents are an established part of the endoluminal treatment of stenoses within various organs, including blood vessels, bile ducts, esophagus, trachea, and bronchi. In particular, metallic stents are commonly used in the treatment of gastric or colonic obstruction, for example to treat obstructions resulting cancerous strictures. Such gastrointestinal stents are not removable except through resection, and generally cannot be repositioned after deployment. The inability to remove gastrointestinal stents has meant that they are primarily useful for patients having a life expectancy of less than a year due to terminal disease, or as a temporary procedure prior to a definitive surgical procedure.

Unlike plastic tubes or catheters, flexible metallic stents have a high ratio of deployed (expanded) diameter to introduction diameter. That is, they have a relatively small-caliber introduction system that allows safe and atraumatic placement via the mouth or anus; yet when deployed, they expand to a diameter large enough to relieve the obstruction. Some of these devices will even pass through the working channel of a therapeutic endoscope.

Gastrointestinal stents have various structural requirements. They must be flexible enough to allow placement but still remain in position once deployed. They need an internal diameter large enough to relieve obstructive symptoms and restore normal eating and bowel habits, and sufficient radial force to expand slowly within areas of fibrosis or neoplasm. They should also prevent obstruction due to tumor ingrowth or reactive hyperplasia. There are covered stents and uncovered stents. Uncovered stents may be more flexible while covered stents are less prone to cancerous tissue ingrowth through the wall of the stent. Both types of stents are subject to hyperplastic tissue ingrowth at either end of the stent with resultant obstruction, and cannot be removed and replaced.

Malignant tumors of the colon and rectum account for significant morbidity and mortality worldwide. A large number of patients having these diseases also have large-intestinal obstruction, and even in those patients who undergo successful resection, recurrent disease may lead to recurrence of intestinal obstruction. The use of stents within the upper and lower gastrointestinal tracts can provide patients palliation of their obstructive symptoms. In addition, the minimally invasive nature of these procedures can allow patients to avoid more extensive and invasive surgical procedures.

However, because the stents are not removable and cannot be easily repositioned, many physicians are hesitant to use them because improper deployment cannot be corrected. In addition, because the stents frequently fail after about a year's time, due to occlusion with debris, they are typically used only in patients who have a terminal condition. There are many patients with other types of strictures that would benefit from a removable stent that could be removed after it has opened up a stricture. The present invention addresses this need for removable stents.

Relevant Literature

The use of gastrointestinal stents is known in the art, for example, see reviews by Soetikno and Carr-Locke (1999) Gastrointest Endosc Clin N Am. 9(3):447-58; or Mauro et al. (2000) Radiology 215(3):659-69.

The patent literature contains descriptions of many different stent designs. A few of the more recent patents include U.S. Pat. No. 5,702,419, “Expandable, Intraluminal Stent”; U.S. Pat. No. 5,707,388, “High Hoop Strength Intraluminal Stent”; U.S. Pat. No. 5,707,387, Flexible Stent”; and U.S. Pat. No. 5,681,345, “Sleeve Carrying Stent”; Palmaz, U.S. Pat. No. 5,102,417, “Expandable intraluminal graft, and method and apparatus for implanting an expandable intraluminal graft”.

The use of endoscopes is well-known in the art, for example a technology status evaluation report may be found in Gastrointest Endosc 2000 Dec; 52(6):864-6; and a review of enteroscopy in Lewis (2000) Gastrointest Endosc Clin N Am. 10(1):101-16, vii. Many U.S. Patents are issued on endoscope designs, the following are included for purposes of illustration: U.S. Pat. No. 6,181,368, Takahashi et al.; U.S. Pat. No. 6,174,280, Oneda et al.; U.S. Pat. No. 6,165,124, Ouchi; and U.S. Pat. No. 6,152,870, Diener.

### SUMMARY OF THE INVENTION

Surgical devices and methods are provided for a system that allows the repositioning or removal of luminal stents, particularly gastrointestinal stents. The system comprises two members: a modified stent and a stent removal device. A conventional stent is modified by the addition of a tightening drawstring with a loop or region for grasping. When the stent is inserted within the body, the drawstring is slack and does not constrict the stent. When the drawstring is tightened, for example by pulling on the region for grasping, the circumference of the stent at the terminus is decreased, allowing the stent to be repositioned or removed without trauma to the organ.

The stent removal device is a flexible elongate member with proximal and distal ends, which can be inserted through a body cavity. Located at the distal end is a grasping member, and a manual means for actuating the grasping member is located at the proximal end of the device. The grasping member is used to grasp the tightening drawstring on the stent.

In one embodiment of the invention, the grasping member can be withdrawn into an elongated sheath, into which the stent may also be withdrawn. The stent removal device may also be contained within an elongated over tube, which over tube can also house an endoscope and other instrument channels.

### BRIEF DESCRIPTION OF THE DRAWINGS

FIG. 1A and 1B illustrate a modified stent comprising a tightening drawstring, in the slack (1A) and constricted (1B) position.

FIGS. 2, 3 and 4 are illustrations of the stent removal device, including a sheath (FIG. 3) and an over tube (FIG. 4).

FIGS. 5A and 5B demonstrate the mechanics of tightening the drawstring on the modified stent, and withdrawing the stent into the over tube.

FIGS. 6A-6D illustrate some embodiments for the tightening drawstring attachment to the stent.

### DETAILED DESCRIPTION OF THE EMBODIMENTS

Surgical devices and methods that permit the replacement or removal of stents are provided. A conventional stent, usually a metallic stent, is modified to permit removal by the addition of a tightening drawstring at the terminus, and optionally over part or all of the length of the stent. When the drawstring is tightened the circumference of the stent is decreased, allowing the stent to be repositioned or removed without trauma to the organ. The stent is removed by tightening the drawstring and withdrawing the stent with a stent removal device, which is a flexible elongate member with a grasping member at the distal terminus, and a manual means for actuating the grasping member at the proximal end of the device. The grasping member can be withdrawn into an elongated sheath, into which the stent may also be withdrawn.

The subject devices find use in a variety of applications where it is desirable to implant a stent. In particular, because gastrointestinal stents are not removable at the present time, their use is generally limited to terminal patients or tissues. The present invention allows an expansion of the patient pool that can be treated with such stents. In further describing the subject invention, the subject devices will be described both in general terms and in terms of representative embodiments depicted in the figures, followed by a review of representative applications in which the subject devices find use and kits that include the subject devices.

Before the subject invention is further described, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

It must be noted that as used in this specification and the appended claims, the singular forms “a,” “an,” and “the” include plural reference unless the context clearly dictates otherwise. Unless defined otherwise all technical and scientific terms used herein have the same meaning as commonly understood to one of ordinary skill in the art to which this invention belongs.

### SYSTEMS AND DEVICES

As summarized above, the subject devices are devices for use in manipulating gastrointestinal stents. As such, the subject devices allow movement of an object inside of a body of an animal. These devices permit the atraumatic removal of stents that have become clogged with debris, or the repositioning of stents that have shifted or were incorrectly positioned. The two components of the system are described below.

### MODIFIED STENTS

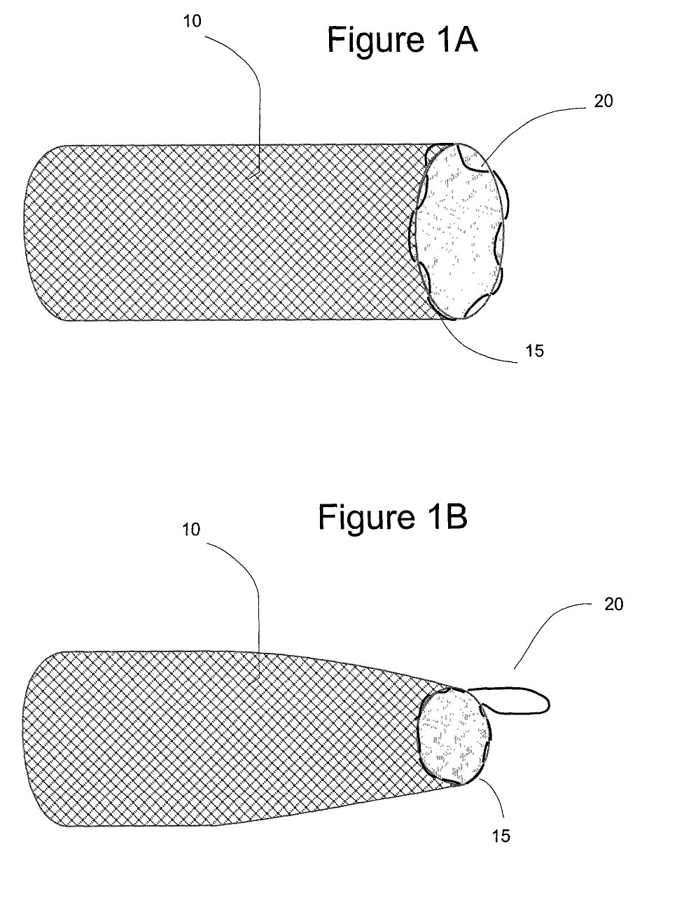

As shown in FIG. 1A and 1B, the stent component of the invention comprises an expandable luminal stent 10, frequently a metallic stent, that has been modified by the insertion of a tightening drawstring 15 that is securely attached to at least one terminus of the stent. The drawstring is a closed circle that is attached to the stent. FIG. 1A illustrates the drawstring in a relaxed configuration, which allows full expansion of the stent, as it resides in the organ. When the drawstring is tightened, as shown in FIG. 1 B, by pulling on a grasping region 20 of the drawstring 15 the circumference of the stent is decreased, usually by at least about 25%, more usually by at least about 50%. Depending on the rigidity of the stent walls, a terminal drawstring may decrease the circumference of the stent along the length.

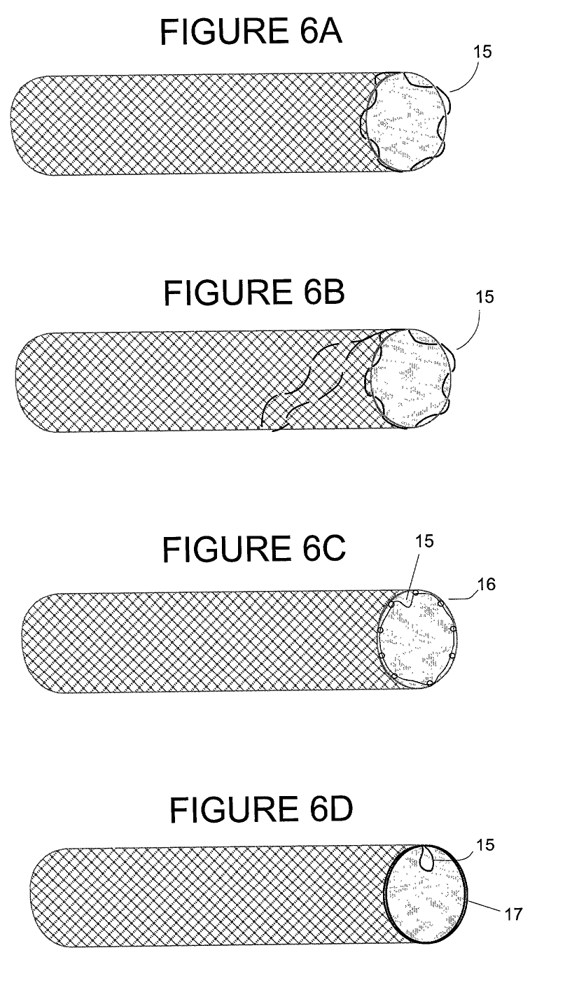

FIGS. 6A to 6D show some examples of suitable drawstring arrangements. In FIG. 6A, the drawstring 15 is threaded through the wires of the stent, leaving a loop for ease of grasping. FIG. 6B shows a drawstring 15 threaded through the length of the stent, so as to reduce the stent circumference more evenly along its length. FIG. 6C shows a drawstring threaded through eyelets 16. FIG. 6D shows a drawstring thread through a flexible annular hoop 17, attached to the terminus of the stent. In other embodiments, a series of drawstrings may be used, distributed along the length of the stent.

The drawstring is typically formed from wire or filament. Filaments may have any cross-sectional geometry, e.g. square, round oval, triangular, etc. The drawstring will be formed from biologically compatible materials, including plastics, such as nylon, stainless steel, titanium, tantalum, gold, platinum, copper and the like, as well as alloys of these metals. The diameter of the filament will be sufficient to provide the flexibility needed for grasping and tightening, and will be sufficiently strong that it will not break during the removal process.

Any stents suitable for use in the gastrointestinal tract and other lumenal organs that do not have the constraints of cardiovascular stents, e.g. to be used in the stomach, small and large intestine, esophagus, tracheobronchial tubes, gall bladder, urinary tract and the like, may be modified by the methods of the present invention. For example, somce commercially available stents include the Gianturco Z-stent (Wilson-Cook, Winston-Salem, N.C.), Wallstent (Boston Scientific, Natick, Mass.), Ultraflex nitinol mesh stent (Microinvasive, Natick, Mass.), and Esophacoil (IntraTherapeutics, Eden Prairie, Minn.). Tracheobronchial stents include the Gianturco Z tracheobronchial tree stent and the Wallstent tracheobronchial endoprosthesis. A Wallstent enteral endoprosthesis is available specifically for the treatment of gastroduodenal and colonic obstructions. The stent may be self-expanding, or may be expandable with a balloon, as is known in the art.

A stent of adequate diameter and length is chosen to traverse the entire stenosis, with allowance of 1-2 cm extending proximally and distally after expansion. If multiple stents are required, the distal stent should be placed initially, with confirmation that it extends well beyond the lesion. At least 1-2 cm of the stents should overlap to prevent migration.

### STENT REMOVAL DEVICE

The stent removal device is a flexible elongate member with proximal and distal ends, which can be inserted through a body cavity. Located at the distal end is a grasping member, and a manual means for actuating the grasping member is located at the proximal end of the device. The grasping member is used to grasp the tightening drawstring on the stent. In preferred embodiments of the subject devices, the grasping member is capable of being retracted into and protruded from a protective housing or sheath located at the distal end of the elongate, e.g. tubular device.

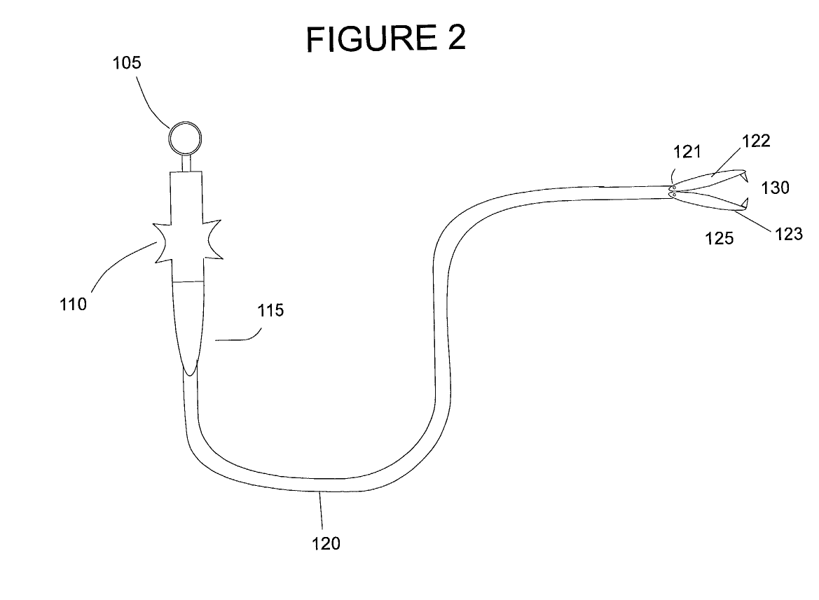

The basic stent removal device is shown in FIG. 2. At the distal end of the flexible elongate member 120 is a grasping device 125. The grasping member typically comprises two or more jaw elements 122 and 123 that can be manipulated to grasp the drawstring. The jaw elements are generally hinged 121 to allow for such movement. In some embodiments of the invention, the jaws will comprise a toothed element 130 for improved grip on the drawstring. In certain embodiments, the device further comprises a jaw element locking means, which serves to lock the jaws in a given position, e.g. in a gripped position.

At the proximal end of the elongate member 120 is a manual means 115 for actuating the grasping member. The manual means may comprise a movable element 105 for opening and closing the grasping member jaws, and a grip 110 for improved control. The manual actuation means may be present in a variety of different configurations, so long as it is capable of providing for the requisite manual control over the movement of the grasping members during use of the device. As such, any manual actuation means that can be operated by hand from a site external to the body and achieve the desired internal object manipulation or movement via the internal articulated member(s) during use may be present on the device. The manual actuation means typically includes one or more elements shaped or configured to be operated by fingers and/or a thumb which are operationally connected to the articulated members via wires, strings, cables, or other tensile elements, etc., to provide for the desired articulate member movement. Manual actuation means of interest include adaptations of those described in U.S. Patents of interest include: 5,997,567; 5,976,122; 5,891,162; 5,820,009; 5,797,959; 5,728,121; 5,713,919; 5,613,973; 5,549,636; 5,417,684; and 5,383,895; the disclosures of which are herein incorporated by reference. Representative manual actuation means are described infra in greater detail.

As mentioned above, the distal and proximal ends of the stent removal device are generally separated by an elongated tubular member. While in many embodiments the cross-sectional shape of the member is curvilinear and most typically circular, other cross-sectional shapes are possible, e.g. square, rectangular, trapezoidal, triangular etc. The diameter of the tubular elongate portion of the device may vary depending on the configuration of the device, but typically ranges from about 0.5 to 10 mm, more usually from about 1 to 5 mm, preferably from about 2-3 mm. The proximal and distal ends are separated by a distance sufficient to provide for the proximal end to be outside of the body and the distal end to be inside of the body during use, where this distance will vary depending on the particular application in which the device is to be used. As such, the distance between the proximal and distal ends in many embodiments of the subject invention generally ranges from about 3 to 60, usually from about 10 to 50 and more usually from about 12 to 50 inches.

The individual elements of the subject devices may be fabricated from any convenient material, where at least the distal portion of the device and elements present at the distal portion are ones that are fabricated from a biocompatible material. Biocompatible materials of interest include biocompatible polymers, where suitable biocompatible polymers include, but are not necessarily limited to: biocompatible polymers and/or elastomers, biocompatible metals as described above, and the like. Suitable biocompatible polymers include, but are not necessarily limited to, materials such as, for example, polyethylene, homopolymers and copolymers of vinyl acetate such as ethylene vinyl acetate copolymer, polyvinylchlorides, homopolymers and copolymers of acrylates such as polypropylene, polymethylmethacrylate, polyethylmethacrylate, polymethacrylate, ethylene glycol dimethacrylate, ethylene dimethacrylate and hydroxymethyl methacrylate, polyurethanes, polyvinylpyrrolidone, 2 pyrrolidone, polyacrylonitrile butadiene, polycarbonates, polyamides, fluoropolymers such as polytetrafluoroethylene and polyvinyl fluoride, polystyrenes, homopolymers and copolymers of styrene acrylonitrile, cellulose acetate, homopolymers and copolymers of acrylonitrile butadiene styrene, polyvinylchloride, silicone rubber, polymethylpentene, polysulfones, polyesters, polyimides, polyisobutylene, polymethylstyrene and other similar compounds known to those skilled in the art. Suitable, biocompatible elastomers include, but are not necessarily limited to, biocompatible elastomers such as medical grade silicone rubbers, polyvinyl chloride elastomers, polyolefin homopolymeric and copolymeric elastomers, urethane based elastomers, and natural rubber or other synthetic rubbers, fluorenated polymers (e.g., PTFE), and the like. The material from which the device is fabricated may include a radiodense material or some other imaging means to allow for visualization, e.g., with fluoroscopy. It should be understood that these possible biocompatible materials are included above for exemplary purposes and should not be construed as limiting.

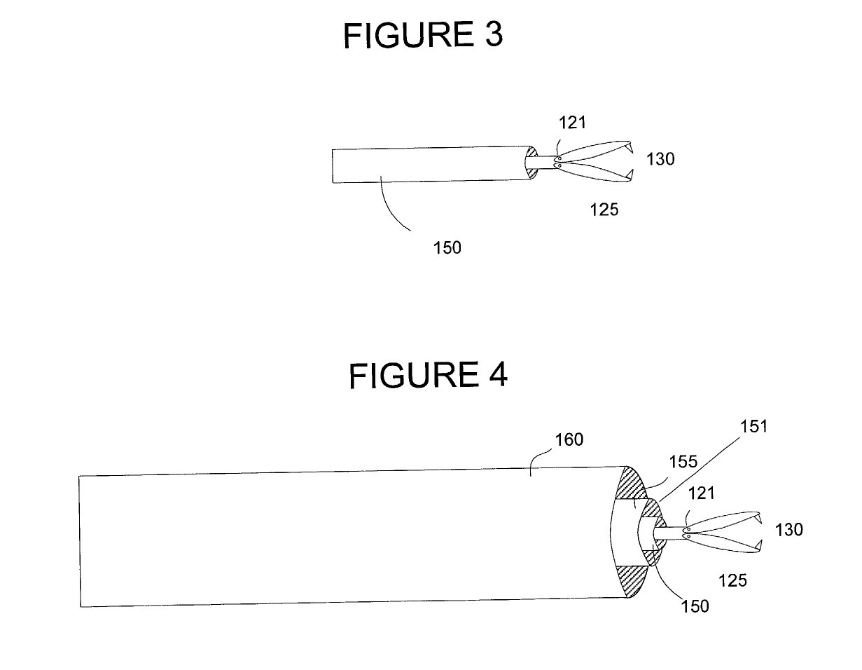

In order to withdraw the stent without damage to the surrounding tissue, it is preferable that the stent removal device comprise a protective sheath, as shown in FIG. 3. The sheath 150 is a protective housing, e.g. a tube, with an opening at the distal end through which the grasping members may be extended or retracted. As such, during introduction of the distal end of the device that includes the grasping members into the body during use, the grasping members or fingers may be retracted into the protective housing to aid in placement of the device at the location of the stent. After the distal end of the device has been positioned at the location of the object to be manipulated, the grasping members may be protruded from the protective housing. Certain flexible stents can be withdrawn into the protective sheath, by grasping the drawstring, and withdrawing the grasping member and stent together into the sheath.

In one embodiment of the invention, the stent removal device is contained within an endoscope tube, as shown in FIG. 4. An endoscope 151 is typically housed in a tube with instrument channels 155, in which tube the stent removal device is housed. Preferably an over tube 160 is used over the endoscope tube. The over tube is of sufficient diameter to permit withdrawal of the stent into the tube. A conventional endoscope has an insertion tube 155 that is connected at a proximal end to a handle or headpiece. The insertion tube often contains an imaging system having optical fibers or the like extending along the length of the insertion tube and terminating at a viewing window in the insertion tube's distal end. The imaging system conveys an image from a viewing window to an eyepiece on the headpiece, or to a monitor, so that the user can see into a selected body cavity during an endoscopic procedure. Through manipulation of the controls, an operator can cause the distal end of the insertion tube to become substantially linear, or to take a curved shape to selectively position the viewing window.

Endoscopes having flexible insertion tubes typically have a flexible outer coating, such as a rubberized material. Protective endoscopic sheaths have been developed to protect insertion tubes from the contaminated external environment, and to protect patients from contaminated insertion tubes. A protective, flexible sheath that is both sterile and disposable can be placed over either a rigid or flexible insertion tube to prevent the insertion tube from being contaminated. After use, the sheath can be discarded. The endoscope can be prepared for the next procedure by merely replacing the sheath with a new, sterile sheath, thereby considerably reducing preparation and down time of the endoscope between procedures.

### METHODS OF INSERTION AND REMOVAL

The stent removal system of the present invention can be used for repositioning or removing stents in the lumen of organs, e.g. stents placed in the gastrointestinal system. The placement of the stent in the organ is performed according to conventional methods, with the exception that the stent itself is modified by the inclusion of a tightening drawstring.

In some cases, stents are found to have been placed in a disadvantageous position, or will shift after the initial placement. Prior to the present invention, it was not possible to reposition the stent without damage to the surrounding tissue. But using the present methods, the stent can be tightened by the drawstring, allowing it to compress in diameter and be correctly placed.

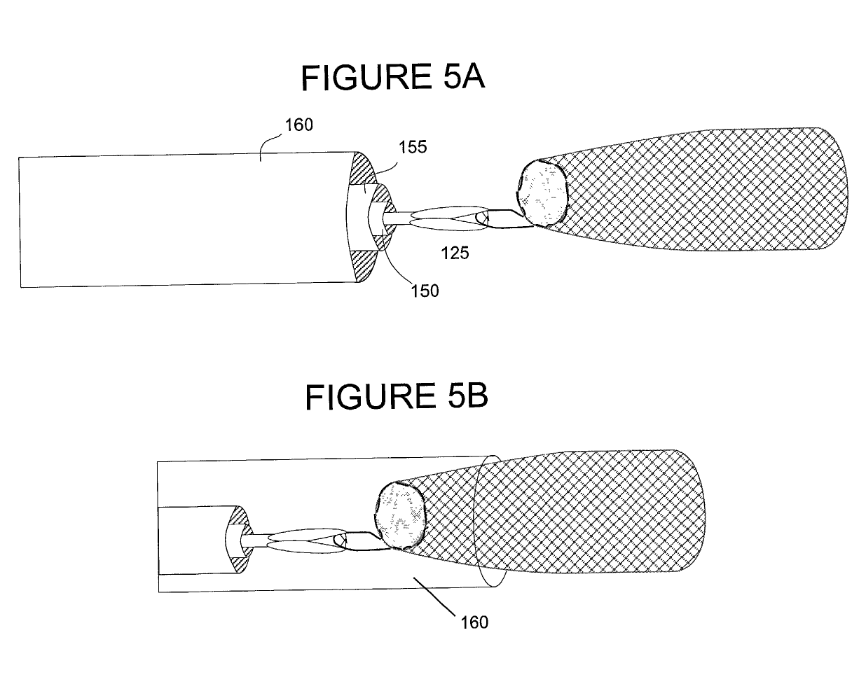

In virtually all cases, a stent in the gastrointestinal tract will become clogged with debris and/or hyperplastic tissue ingrowth, and will generally need to replaced after about a year of use. As previously discussed, this need for removal and/or replacement has severely limited the use of stents, even where they can provide useful treatment. A general method for repositioning and removal is described below, and is shown in FIG. 5.

The stents can be placed and removed/repositioned with fluoroscopic guidance, endoscopic guidance, or a combination of both. Fluoroscopic monitoring is extremely helpful in accurately deploying and removing the stent, where, for example, iodinated contrast material is injected with a catheter to identify the proximal extent of the stricture. Once a catheter has been passed beyond the lesion, contrast material is injected again to demonstrate the distal margin of the stenosis.

Access to the stent can be obtained through the nasogastric tube, e.g. using a steerable angiographic catheter, which is passed via the nose or mouth. The catheter is manipulated by using standard catheter and guide wire techniques. Alternatively, access can be achieved with percutaneous gastrostomy or endostomy by using standard techniques.

Endoscopic guidance adds increased control, which facilitates both catheterization of the stent and accurate device removal. The stent is first identified endoscopically; then, the stent is grasped with the jaw of the grasping element. The grasping element is withdrawn into the endoscope or the over tube, thereby tightening the drawstring, and at the same time both decreasing the circumference of the stent, and removing it from the tissue, to pass the stent either within the sheath of the stent removal device, or into the over tube surrounding the endoscope, as shown in FIGS. 5A and 5B. Once the tightening drawstring 20 is grasped by the grasping device 125, the sheath 150 is pushed out until it covers the grasping device 125. Then the grasping device and stent is pulled into sheath 150. The overall result is that 150 is positioned right up against the looped part of the drawstring and the stent collapses into a cone with the tip of the cone at the opening in 150. The process can also be monitored fluoroscopically. Contrast material may be injected to confirm successful removal of the stent. and fluoroscopic

### UTILITY

Malignant obstruction of the stomach, eosophagus, or duodenum causes nausea, vomiting, esophagitis, electrolyte imbalance, poor nutrition, and severe dehydration. Causes include primary tumors of the stomach and duodenum, malignant infiltration by neoplasms from adjacent organs (eg, pancreas), and compression by malignant regional lymphadenopathy. Curative resection is not possible in 40% of patients with gastric cancer and 80%-95% of patients with pancreatic cancer. Expandable stents offer a nonsurgical alternative for treatment. These are particularly useful in poor surgical candidates with malignant obstruction of the gastric outlet. The ability to treat duodenal obstruction secondary to extrinsic compression from pancreatic cancer further expands the spectrum of indications of stent placement for primary nonsurgical palliation. Stents have also been used in patients with benign gastroduodenal strictures when conventional surgical resection or bypass was not possible or wanted. In patients with benign disease, coexistent morbid factors involving the cardiopulmonary systems may limit surgical options, which makes the use of metallic stents more attractive. In all cases, the ability to reposition or remove stents after placement expands the candidate pool for this treatment.

### KITS

Also provided are kits that at least include the subject devices, for a modified stent, or drawstring suitable for stent modification, and a stent removal device. The subject kits further include instructions for how to use the device in a procedure. The instructions are generally recorded on a suitable recording medium. For example, the instructions may be printed on a substrate, such as paper or plastic, etc. As such, the instructions may be present in the kits as a package insert, in the labeling of the container of the kit or components thereof (i.e. associated with the packaging or subpackaging) etc. In other embodiments, the instructions are present as an electronic storage data file present on a suitable computer readable storage medium, e.g. CD-ROM, diskette, etc. The instructions may take any form, including complete instructions for how to use the device or as a website address with which instructions posted on the world wide web may be accessed.

What is claimed is:

1. A method for the repositioning or removal of a luminal stent, the method comprising:
modifying a luminal stent prior to insertion in a tissue, by adding a tightening drawstring through one or both termini of the stent, wherein said drawstring is slack during normal use of the stent;
repositioning or removing said stent by the method of:
grasping said drawstring with a grasping element of a stent removal device;
withdrawing said grasping element into a protective sheath or over tube, thereby tightening said drawstring and decreasing the circumference of said stent;
withdrawing said stent at least partially into said protective sheath or over tube in order to reposition within said tissue or to remove from said tissue;
withdrawing said stent removal device.

2. The method according to claim 1, wherein said luminal stent is inserted in the gastrointestinal tract.

3. The method according to claim 1, wherein said stent removal device is housed with an endoscope.

4. The method according to claim 3, wherein an over tube is provided to cover said endoscope, and wherein said stent is withdrawn into said over tube.

5. The method according to claim 1, wherein said stent is a metallic, self-expanding stent.

6. The method according to claim 1, wherein said tightening drawstring is a nylon filament.

7. The method according to claim 1, wherein said tightening drawstring is threaded through the wires of said stent at one terminus.

8. The method according to claim 1, wherein said tightening drawstring is threaded through the wires through at part of the length of the stent.

9. The method according to claim 1, wherein said tightening drawstring is threaded through eyelets provided on the terminus of the stent.

10. A stent removal system, the system comprising:
a luminal stent comprising a tightening drawstring through one or both termini of the stent, wherein said drawstring is slack during normal use of the stent;
a stent removal device, comprising a flexible elongate member with proximal and distal ends, a grasping member at the distal end, and a manual means for actuating the grasping member at the proximal end of the device.

11. The system according to claim 10, wherein said stent removal device comprises a protective sheath over said elongate member.

12. The system according to claim 10, wherein said stent removal device further comprises an endoscope, and a protective over tube.

13. The system according to claim 10, wherein said stent is a metallic, self-expanding stent.

14. The system according to claim 13, wherein said stent is suitable for gastrointestinal use.

15. The system according to claim 10, wherein said tightening drawstring is a nylon filament.

16. The system according to claim 10, wherein said tightening drawstring is threaded through the wires of said stent at one terminus.

17. The system according to claim 10, wherein said tightening drawstring is threaded through the wires through at part of the length of the stent.

18. The system according to claim 10, wherein said tightening drawstring is threaded through eyelets provided on the terminus of the stent.
